(19) United States
(12) Patent Application Publication
(10) Pub. No.: US 2011/0213281 A1
(43) Pub. Date: Sep. 1, 2011

(54) METHOD AND APPARATUS FOR THE REMOVAL OF NON DESIRED BIOLOGICAL COMPONENTS FROM SURFACES OF EXTERNAL TISSUES

(76) Inventor: Yoni Iger, Haifa (IL)

(21) Appl. No.: 13/036,100

(22) Filed: Feb. 28, 2011

Related U.S. Application Data
(60) Provisional application No. 61/309,349, filed on Mar. 1, 2010.

(51) Int. Cl. A61N 7/00

(52) U.S. Cl. 601/2

(57) ABSTRACT

The present invention provides a method, device and system for the removal of non-desired biological components from external surfaces of tissues. This, in accordance with the invention, is achieved utilizing ultrasound device and ultrasound-derived mechanisms employed essentially externally to the external surfaces of tissues to be affected, and while keeping the said tissues intact with respect to the impact of the bulk of the affected ultrasound.

The method, in accordance with the invention, comprises accordingly exposing the supportive medium coupling between the ultrasonic emitting source, and the external surfaces to be affected, to a relatively low frequency ultrasound stimulus emitted at certain angle with respect to the surfaces to be affected, whereas said stimulus being such as to create essentially non-thermal ultrasonic derived phenomena in the said coupling medium for impacting the external surfaces of the desired tissues.

The system of the invention generally comprises elements supporting formation of the desired ultrasound phenomena, and at desired location and angle. It is comprised of control unit, a single or a multi-frequency signal generator, a signal amplifier, a matching unit, treatment chamber, and at least one transducer. Occasionally multiple transducers, or multi-frequency transducer, or transducers of different types including phase array, may be used to create different effects at pre-determined locations of external surfaces, where treatment is conducted in treatment-chamber via coupling medium. The system therefore further contains coupling medium, in which phenomena are created and through which impact is performed onto the external surfaces of tissue.

### RELATED APPLICATIONS

This non-provisional patent application claims priority to U.S. provisional patent application Ser. No. 61/309,349 filed Mar. 1, 2010, the disclosure of which is incorporated herein in its entirety by reference.

### FIELD OF THE INVENTION

The present invention concerns a method, device and system for the removal of non desired biological components from external surfaces of tissues, using ultrasound-derived mechanisms employed essentially externally to the tissue, and while isolating the bulk of the tissues from the impact of the affected ultrasound.

### BACKGROUND OF THE INVENTION

Systems and methods for affecting biological tissues using ultrasound energy are well known. U.S. Pat. No. 7,828,734 of Azhari et al., describes an apparatus composed of plurality of transducers adapted to transmit energy towards each other and into determined tissue located in between. U.S. Pat. No. 5,601,526 of Chapelon et al, describes apparatus for applying ultrasound waves focused onto a focal point in the body using thermal and cavitation modalities. U.S. Pat. No. 6,500,141 of Irion et al., describes an apparatus for treating superficial body tissues, by applying ultrasound from an applicator facing the body surface through the surface and into the body tissue.

Surfaces of tissues may suffer non desired proliferation or growth, or accumulation of non desired biological components due to intrinsic or extrinsic factors. For example, in external tissues directly exposed to the external environment there might be accumulation of melanin forming pigmented lesions, or of stratum corneum components forming plaques of dry cellular membranous material on the skin surface in regular or in psoriatic patients; there might be fibrovascular scar-outgrowth of conjunctiva and blood vessels, or yellowish outgrowth of the conjuctiva (pterygium and pinguecula) on UV-exposed surface of the eye, or need to remove areas of cornea for vision correction. Similar phenomena of non-desired proliferation or growth, or accumulation of non-desired biological components may occur also in external surfaces of deeper located tissues; there might be excessive accumulation of blood in the uterus endothelium and excessive menstrual bleeding in particular at early stages of menopause; there might be polyps growth into the intestine lumen and accompanied gastro-bleeding or intestine blockage; there might be intrusion growth of tissue into the bladder hampering ability to urine, or benign or cancerous prostate, or cancerous tumor growth on the interior (external) surface of the pleura.

Attempts were made for removal of materials from surfaces of tissues. For example, US patent application publication 2002/0000763A1 of Jones describes methods for selectively dissolving and removing solid and semi-solid materials from surfaces of soft tissue such as the blood vessels or the prostate lumen, using ultra-high frequency ultrasound of 50 MHz to 100 GHz. According to that method, ultrasonic waves are applied to the unwanted material via a zone of acoustic mismatched impedance, while at least one emitting transducer is in contact with the unwanted material, and the applying involves repeatedly rubbing of the active face of the transducer across the surface of the unwanted tissue for the removal of material therefrom.

At times, it is desirable to remove biological components from external surfaces of tissues for their resurfacing, while minimizing the energy delivery to the said tissues, therefore minimizing the non-desired or non-predicted impact employed to these tissues. It is also desired to do such removal regardless of the biological material to be removed, or the external surface of tissue to be affected. It is also desirable to make it in a precise way, so to remove volumes of tissues at sub-incision resolutions of tens or hundreds of microns, and at different volumes of tissue removal.

It is the object of this invention to provide a method and device for removal of non-desired biological components from external surfaces of tissues.

### SUMMARY OF THE INVENTION

In view of the above, the present invention provides a method, device and system for the removal of non-desired biological components from external surfaces of tissues. This, in accordance with the invention, is achieved utilizing ultrasound device and ultrasound-derived mechanisms employed in proximity to, still essentially externally to, the external surfaces of tissues to be affected, and while keeping the said tissues intact and opaque with respect to the impact of the bulk of the affected ultrasound.

The method, in accordance with the invention, comprises accordingly exposing the supportive medium coupling between the ultrasonic emitting source, and the external surfaces to be affected, to a relatively low frequency ultrasound stimulus emitted at certain angle with respect to the surfaces to be affected, whereas said stimulus being such as to create essentially non-thermal ultrasonic derived phenomena in the said coupling medium for impacting the external surfaces of the desired tissues.

The ultrasound derived phenomena or mechanisms of impact on external surfaces of tissues may be cavitation and derived micro-streaming and micro-shear stress, acoustic streaming, stirring and rotation, torque, radiation force, pressure, shock waves, shear stress and the like. The impact of these phenomena is perforation and disintegration of cell membranes and entire cells, till layers of cells, from the external surfaces of the affected tissues.

It should be noted that for the purpose of the current invention “external surfaces of tissues” is determined as boundary layers at surfaces of tissues. Organs having tissues with external surfaces may be located in proximity to the exterior environment, for instance the skin, tonsils, or the cornea. Tissues with surfaces may be deeper located tissues, having direct or indirect anatomical connection with the exterior environment, for instance the digestive tract, respiratory, urology or reproductive systems. For instance the lumen of the uterus is connected via the vagina to the exterior environment, and the lumen of the bladder or prostate is connected via the urethra to the external environment. However, internal organs having lumen, have accordingly two external surfaces tissues—at the outer cover tissues of these organs, as well as the inner most surface facing the lumen of theses organs. Tissues with surfaces may also be for instance the pleura having lamina with external surface facing the lungs, or the kidney having capsule-like structure with external surface.

At times there may be temporal change and exposure of interior parts of tissues, normally not functioning as boundary layer, as external surface. For instance during LASIK procedure for vision correction there is a temporal removal of flap of the cornea followed by ablation of internal parts of the cornea that now become exposed to the exterior environment. At that stage of exposure, this is also considered as external surface of tissue for the purpose of the current invention. Also, for the purpose of the current invention, post removal of external surfaces of tissues, the newly exposed tissue that forms new external surface of said tissue, is also considered as external superficial surface of tissue and may further be treated as such using teaching of the invention.

For the removal of non-desired biological components from external surfaces of tissues, ultrasound coupling medium shall be essentially located between the ultrasound emitting end of the ultrasound source and the surfaces to be affected. Said coupling medium shall be chosen according to the desired effect, it may be liquid when e.g., cavitation is desired, or may be medium of high viscosity when e.g., sheer stresses are desired. The coupling medium may be of exogenous or of endogenous origin. For instance when liquid coupling media is to be used, it may be medium of exogenous origin, e.g., water or physiological solution for treating the skin or surfaces of internal tissues respectively, or medium of endogenous origin, e.g., urine as coupling medium when treating external surface of the bladder (facing the lumen), or intestine mucus when treating digestive tract.

According to a non-limiting embodiment, by emitting the ultrasound waves at a certain angle beyond critical angle to reflection with respect to the tissue, impinging ultrasonic waves are reflected from the surface and essentially do not penetrate the tissue. Under such conditions, ultrasound-derived phenomena will occur at predetermined location external to, still proximal to the surface of the affected superficial tissue.

According to yet another non-limiting embodiment of the invention, ultrasonic waves may be applied to and propagate in the coupling medium in parallel and in proximity close enough to enable effect to the external surfaces of tissues exposed.

According to yet another non limiting embodiment of the invention longitudinal or shear waves may also be applied to both external surfaces of exposed tissues as surface waves, as well as to the coupling medium surrounding it, either while reflecting therefrom or in parallel to the said surfaces. Since different minimal thresholds of impact exist between tissue and coupling medium for defined ultrasound derived mechanisms, using appropriate parameters desired impact can be performed only externally for these tissue surfaces. For instance, since there is different threshold for cavitation in aqueous medium, versus threshold of cavitation in tissues, applying ultrasound energy under this embodiment, using the appropriate parameters and via appropriate coupling medium, will enable cavitation outside of the external surfaces of affected tissue, and impact performed on surface.

According to yet another non limiting embodiment, emitting the ultrasound waves at a certain distance from the tissue enable creation of e.g., energy maxima, either per-se or while using HIFU, in proximity and having beam path essentially parallel to the external surface, enabling creating desired ultrasound derived effect external to the surface and actual impact therefrom.

According to yet another non limiting embodiment of the invention, the use of combination of ultrasound sources, angles, directions, frequencies and created ultrasound-derived phenomena is enabled, for instance by means of multiple-frequency transducer or transducers or phase-array source. According to this embodiment using for instance phase array modality, portion of which is used for creating cavitation in parallel and close proximity to the surface to be affected, then before collapse of cavitation boundary that also isolate delivery of ultrasound emitted at angles to the body, there is emission from a second ultrasonic source (transducer of different component of phase array) that creates similar or other phenomena in the coupling medium close to the surfaces of the external tissues exposed.

The device of the invention can be constructed either as ultrasound-delivery device for superficial tissues, or as catheter-like or an endoscope-laparoscope-like device for affecting surfaces of deeper located external tissue in minimally invasive procedures.

The device can be operated as stand alone device, for instance during external procedures; It can be further operated in conjunction with other devices, for instance monitoring devices. The device can utilize the ultrasound emitting source, or other ultrasound source, or any other monitoring mean for instance OCT or optic fiber, to monitor the impact of treatment and accordingly to decide on progress of treatment and on continuation or discontinuation of treatment according to impact achieved.

Generally speaking, the method of the current invention require performing phenomena characterized by high mechanical index versus low thermal index in close proximity to tissue. The ultrasound stimulus shall have the following parameters: Frequency: At least 20 kHz and up to 10 MHz, most preferably, 20 kHz to 1 MHz; Mode: continuous or disrupted of different duty cycles; Duration: at least half of a cycle; Intensity: 0.01-5,000 W/cm², most preferably 10-500 W/cm²; Modality—single or several wave-lengths. Under preferred yet non-limiting embodiment, the ultrasonic stimulus force is used to cause cavitation in proximity to the external tissue to be affected.

The method of the present invention may be used for therapeutic and for cosmetic purposes, as well as for diagnostic and experimental purposes. It may for instance be used for removal of external portions of tissue for diagnosis, for fractional or complete resurfacing, for removal of lesion, for reshaping of structure and the like. Occasionally, surfaces of external tissues might pass pre-treatment before employing method and device of current invention, for instance to increase their susceptibility to the ultrasound-derived mechanisms. At times, external surfaces of external tissues might pass post-surfaces-removal treatment, for instance by applying occlusion dressing post skin rejuvenating resurfacing. Also, method of current invention may be used as pre-treatment, for instance before drug delivery via external surfaces of tissue or for wound debris-removal before application of medication. It may also be used as post treatment modality, for instance for removal of remnants of HOLAP/HOLEP ablation process from prostate, or for the removal of debris of ablated tissue post laser skin resurfacing.

### BRIEF DESCRIPTION OF THE DRAWINGS

FIG. 1 shows a schematic drawing of an ultrasound device of the invention used to remove pterigium from external surface of the cornea.

FIG. 2a shows a schematic drawing of an ultrasound device of the invention, used to remove biological components from external surface of large area, for instance skin for skin rejuvenation.

FIG. 2b shows a shematic drawing of a phase array component of FIG. 2a, enabling fractional treatment.

FIG. 3 shows a schematic drawing of hand-held ultrasound device of the invention, used to remove biological components from external surfaces.

FIG. 4 shows a schematic drawing of a multiple-source ultrasound device of the invention, used to remove biological components from external surfaces of tissues.

FIG. 5 shows a schematic drawing of a minimally invasive ultrasound device of the invention used to remove biological components from external surfaces of deeper located tissues in lateral emission.

FIG. 6 shows a schematic drawing of a minimally invasive ultrasound device of the invention used to remove biological components from external surfaces of deeper located tissues in front emission.

### DETAILED DESCRIPTION OF THE INVENTION

A device in accordance with the invention, functions to remove biological components from external surfaces of tissues, using ultrasound to create ultrasound derived phenomena essentially external and superficial to the external surfaces of tissues to be affected, and related ultrasound derived impacts essentially in the external surfaces of the desired tissues for removal of biological components therefrom. Said device essentially does not enable the bulk of delivered energy to impact deeper tissue at location of removal of biological component.

The system of the invention generally comprises elements supporting formation of the desired ultrasound phenomena, and at desired location and angle. It is comprised of control unit, a single or a multi-frequency signal generator, a signal amplifier, a matching unit, treatment chamber, and at least one transducer. It shall be noted that occasionally multiple transducers, or multi-frequency transducer, or transducers of different types including phase array, may be used to create different effects at pre-determined locations of external surfaces, where treatment is conducted in treatment-chamber via coupling medium. The system therefore further contains coupling medium, in which phenomena are created and through which impact is performed onto the external surfaces. Said coupling medium may be exogenic or body-endogenic, and may be of different viscosities according to the phenomena to be created. Coupling media may be enriched by enhancers, for instance micron or sub-micron particles functioning as cavitation-grains for external use, or CO₂ gas for gas-enrichment liquid environment in endogenous use. Coupling medium may function also as cooling agent.

The geometrical dimensions of the device, the emission incidence and impingement angle, the desired impact and the required coupling medium, shall be calculated or measured and determined according to the requested desired effect, and may be monitored during process. Monitoring elements, for measuring and stabilizing angle between emitting element and affected surfaces, or for measuring actual tissue structure before and during impact on tissue may also be incorporated into the delivery system, or be activated in parallel to the system. The device may be further or alternatively equipped with automatic means for aligning ultrasound emitting angle with actual angle of surface, for instance using an array of ultrasound reflectors located on flexible sheet, curve of which is determined by external surface on which it is placed.

The system of the invention can be constructed either as external device for superficial tissues, or as an endoscope-laparoscope-like or catheter-like device for affecting external surfaces of deeper located tissues, for instance in minimally invasive procedures.

The device of the invention can be operated in a manual mode or in automatic mode, for instance in a scan-like mode according to a predetermined plan or according to integration of feedback of the monitoring element of the system, and can enable complete or fractional treatment.

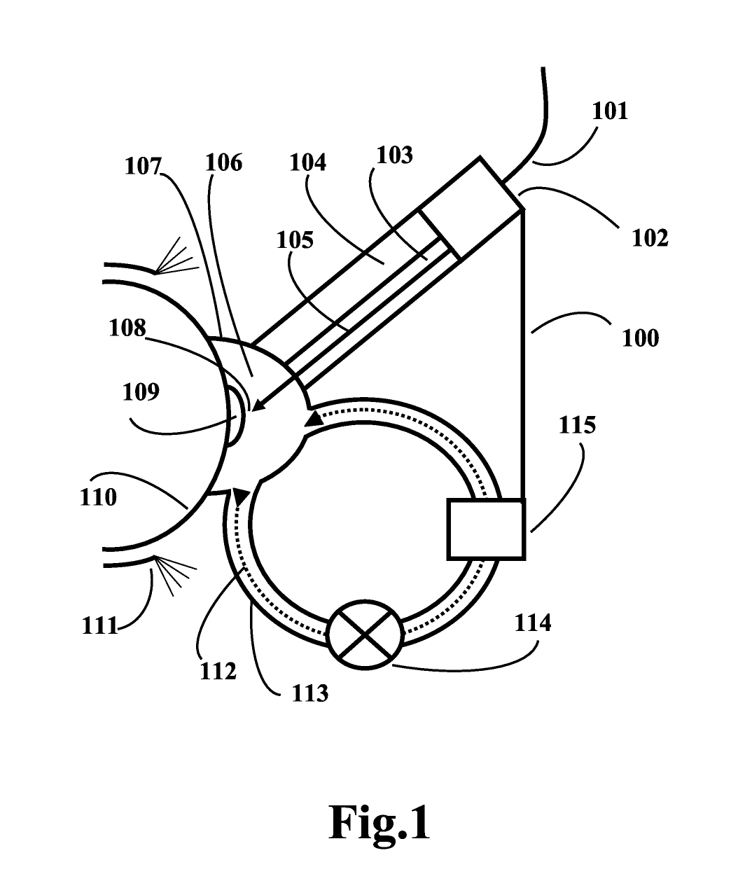

A conceptual model of the delivery device 100 in accordance with the invention, is shown in FIG. 1. The delivery device 100 is operated by electricity, and is part of a system composed of control unit, a signal generator, a signal amplifier, and matching unit (not shown) which are connected to the ultrasonic emitting element 102, for instance a piezoelectric element, via communication cable 101 that includes an appropriate electrical source. The delivery device according to this non limiting drawing is used to remove pterigium out-growth 109 from the surface of cornea 110, distantly from eyelash 111. The system 100 is attached to the cornea via treatment-chamber 107, of which volume 106 is filled with coupling medium. Before operation, monitoring element 104, measures the curvature appearance and distance of distal surface of 109 and communicates the control unit (not shown) via communication cable 101 to accordingly establish angle of emission, for instance using motorized-transmission. This may be done for instance by changing orientation of emitting source 102 versus target, for instance when regular emitting element is used, or by controlling the activation of certain components of emitting element 102, for instance when a phase array element is used. The monitoring and fitting may be conducted continuously during treatment. Ultrasound waves are emitted from distal part of ultrasonic emitting element 102, via ultrasonic guide 103 composed of coupling medium. It should be noted that ultrasonic emitting element 102 may be located also in the system, so not in the actual delivery device, providing having appropriate ultrasonic guide 103 that deliver the ultrasonic energy from its source 102 to its target 109. Waves in the general direction of arrow 105 further propagate into treatment chamber 107 attached to cornea 110, having the said pterigium out-growth 109 growth 6, for its removal. Emitted waves impinge pterigium 109 on its external surface 108 at a certain predefined angle. At that point 108 for instance maxima of the ultrasonic waves exist and accordingly high intensity streaming and cavitation occur fragmenting the surface of pterigium 109 for its removal by continuously removing external surfaces of it. The delivery device 100 also include mini-pump 114 used to circulate coupling liquid Accordingly, coupling medium of volume 106 is pumped via tube 113, in the direction of schematic dashed arrow 112 further into filter 115 from which it is recycled back into treatment chamber 107 in the general direction of dashed arrow 113. The filtration enables keeping the liquid viscosity and the desired impact constant, albeit the cellular additives from the fragmented pterigium, therefore enabling controlled streaming or cavitation without the need to adjust parameters due to released debris fragments. Before and during operation, monitoring element 104 which may include for instance OCT, may be used to determine delivery angle versus surface, or stop point, when sufficient amount of external surfaces has been removed. Ultrasound element 102 may also be used to determine the structure or the curvature appearance of cornea 110 before and during treatment. Post employing that teaching of the invention, post treatment, for instance supportive medicine, may be applied to the treated zone.

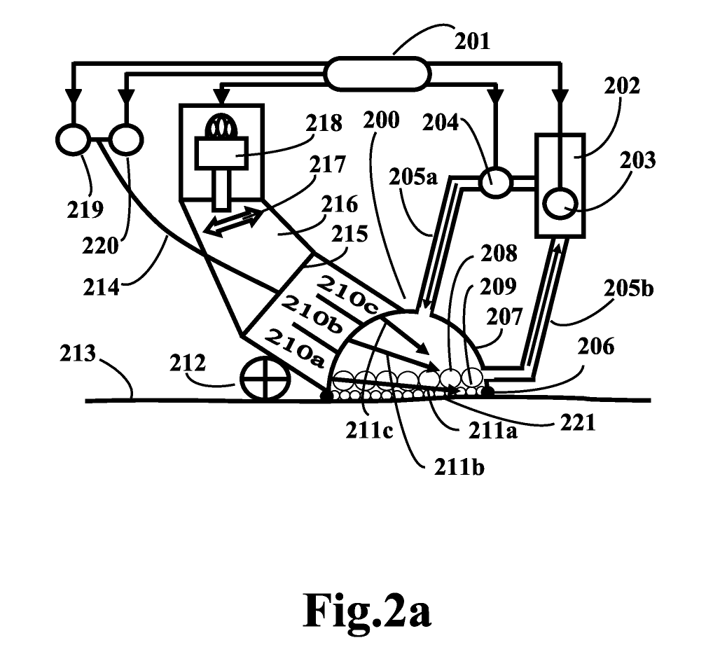

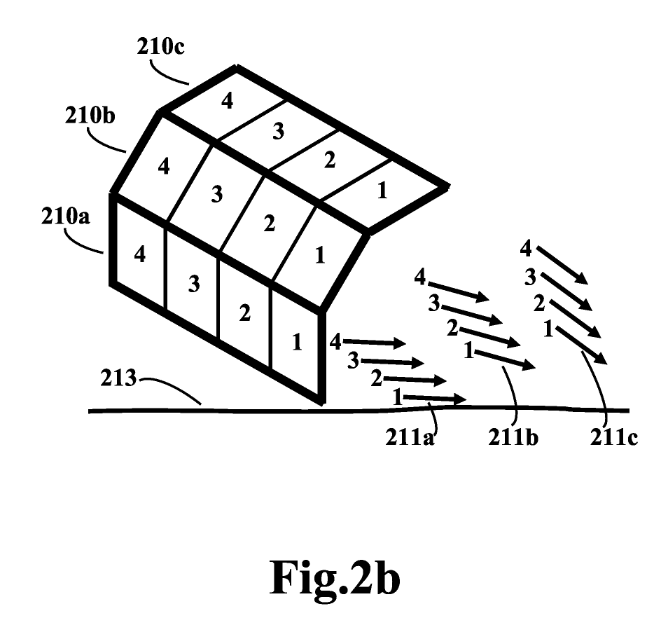

FIG. 2a discloses delivery system 200 for the removal of non-desired biological component from external surfaces of tissues. Treatment chamber 207 of system 200 engulfs coupling medium, is located on external tissue surface 213, to which it is attached via mean 206, for instance a rubber ring or vacuum-enabling tube. The ultrasound emitting element 215 of this system 200, is composed of a phase array element, of which three emitting components 210a, 210b and 210c, each located at different angle with respect to treatment zone, are presented. The ultrasound emitting element 215 is communicated and activated via communication cable 214. It is actuated by controller 201, and may also be aligned via phase-array micro-controller 219, post integration of input of tissue monitoring element 220.

Post establishing of appropriate angle and distance of ultrasound emitting element 215, using monitoring and angle establishing mean 216, ultrasound is emitted. Phase array element 210a emit ultrasound in close proximity and essentially parallel to treatment area 221, in general direction of arrow 211a. This create cavitation bubbles 209 in close proximity to treatment area 221. Subsequently and within period of existence of bubbles 209, phase array element 210b emits ultrasound of different angle and higher frequency in general direction of arrow 211b to create cavitation bubbles 208 in close proximity to bubbles 209, and while cavitation bubbles 209 functions as barrier in preventing the passage of ultrasound waves 211b there-through and into the tissue 213.

Concomitantly, or subsequently depending on plan, phase array element 210c emits ultrasound in the general direction of 211c. That emission is blocked from entering the skin by passage-preventative impact of bubbles 209 and bubbles 208, and is used for instance to create acoustic pressure, pushing the cavitation bubbles 209 and 208 toward the treatment zone 221. Using pump 203 the affected coupling medium, which includes cellular debris removed from treatment area 221, may be further pumped out of treatment chamber 207 via tube 205b, in the general direction of the arrow and into coupling medium reservoir 202. It may then be cleaned off the debris impurities in filter 204, from which it may be recycled into treatment chamber 207 via tube 205a, in the general direction of the arrow in said tube. These activities of pumping and filtration may also be controlled by controller 201, and slightly excessive pumping via tube 205b may be used as a suction-creating mode derived from negative pressure created in treatment chamber 207, enabling good attachment as well as possible deformation of treatment zone 221, to enable appropriate angle of impingement of the ultrasound waves.

The system 200 may be removed over treatment area 213 with the support of moving element 212, for instance a mini-wheel. Element 212 may further contain means for continuous movement over the desired location, and may also function as encoder to detect location and areas to which treatment has already been employed, or as monitoring element supporting activity of 220.

At times, system may also include scanning element 218, that may function in mechanical shifting and positioning of the components of ultrasound emitting element 215 with respect to tissue 213, for instance in direction of arrow 217. Element 218 may further be electronically coupled with phase array micro-controller 219 and function as electronically acoustic scanner, enabling a controlled two dimensional screening and impacting of treated area 221, above its capability to align angle.

FIG. 2b describes further possible arrangement of emitting sub-elements, for instance transducers, of phase array 210a to 210c of FIG. 2a. According to teaching of FIG. 2b, each of phase array elements 210a, 210b and 210c may be further composed of sub-elements 210a1 to 210a4, 210b1 to 210b4, and 210c1 to 210c4 respectively, emitting energy in sub-directions of 211a1 to 211a4, 211b1 to 211b4, as well as 211c1 to 211c4 respectively. The scanning modality capability described above in relation to elements 218 and 210 of FIG. 2A, enables precise impact per treated zones. So for instance if a more delicate cavitational impact is required per sub-zone of treatment then relevant portion of the 210b elements is not activated; if groove exist in treatment surface then relevant portion of the 210c elements is activated at higher intensity to enable better pushing or coupling of the cavitation bubbles and the treatment zone. It accordingly the sub-elements design and its control also enables performing complete as well as fractional treatment of external surfaces of tissues.

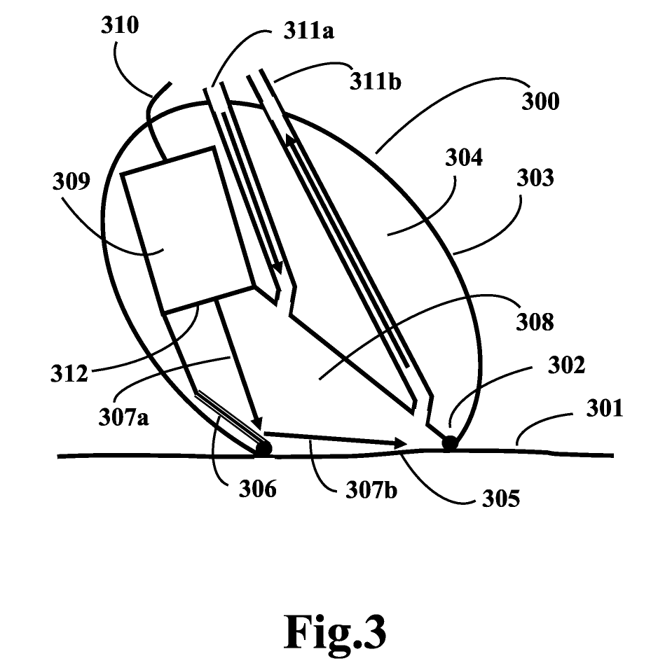

FIG. 3 discloses delivery system 300 attached to external surface 301 via mechanical coupling means ring 302 that may include suction. Housing 303 includes ultrasonic isolated area 304 composed for instance of air, and treatment chamber 308 with acoustic coupling medium that may be circulated out and returned back via tubes 311b and 311a in the general direction of the schematic drawn arrows. Ultrasonic emitting surface 312, of ultrasonic emitting element 309 is connected to controller (not seen) via communication cable 310 and emits ultrasound waves via coupling medium of treatment chamber 308 in the general direction of arrow 307a. Said ultrasonic waves impinge ultrasonic reflector 306, and reflected waves further propagate in the general direction of arrow 307b to affect for the removal of non-desired biological components from the external surface of treatment area 305. Impact of waves in general direction of arrow 307b, may for instance be streaming, using the movement of regular or particle-enriched coupling medium for controlled rubbing impact on exposed external surfaces. It shall be noted that ultrasonic reflector 306, may be located on flexible matrix, so with different pressure, or due to different surface structure, it may be disposed, enabling automatic change and alignment of impinging waves with respect to needs.

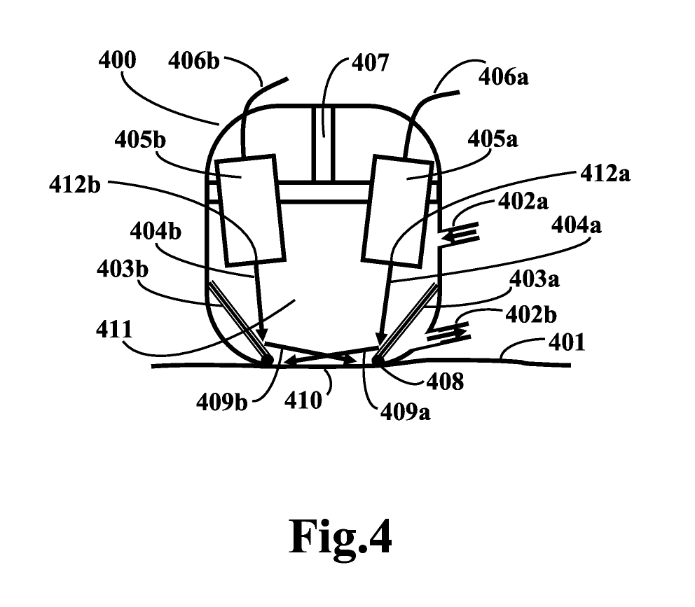

FIG. 4 discloses delivery system 400 composed of more than one ultrasound emitting element. According to this example of system 400, two ultrasound emitting elements 405a and 405b, are communicated via cables 406a and 406b respectively, to impact external surface 401. The ultrasound emitting elements are located at a certain predetermined fixed position inside delivery system 400, using support 407. System 400 further include treatment chamber 411, with coupling medium that may be out and in circulated via tubes 402b and 402a respectively, in the general direction of the drawn arrows. During operation, ultrasound emitting element 405a emits via irradiation surface 412a ultrasound waves in the general direction of arrow 404a. said waves impinge acoustic reflector 403a and propagate towards treatment area 410 at certain predetermined angle in the general direction of arrow 409a. Angle of propagation to enable appropriate impact according to the invention, may be determined for instance by the angle of the transducer 405a, or angle of any of its emitting elements 412a, or by controlled activation of certain predetermined sub-zone emitting elements if for instance phase array is used, or by the angle of the acoustic reflector 403a, and their combinations. At certain time interval, before, concomitantly with or after the activation of ultrasound emitting element 404a, ultrasound emitting element 405b is activated to emit via irradiation surface 412b ultrasound waves in the general direction of arrow 404b. Said waves impinge acoustic reflector 403b and propagate towards treatment area 410 at certain predetermined angle in the general direction of arrow 409b. At treatment zone 410 cumulative impact may be performed due to the integrated vectorial contribution of ultrasonic waves in the intra-coupling zone of arrows 409a and 409b. For instance different modalities of impact may be enabled there, or due to the use of different frequencies or time coupling between emissions, constructive intra-coupling effects may be enabled for instance for lowering thresholds of desired ultrasonic phenomena and increasing overall yield of treatment.

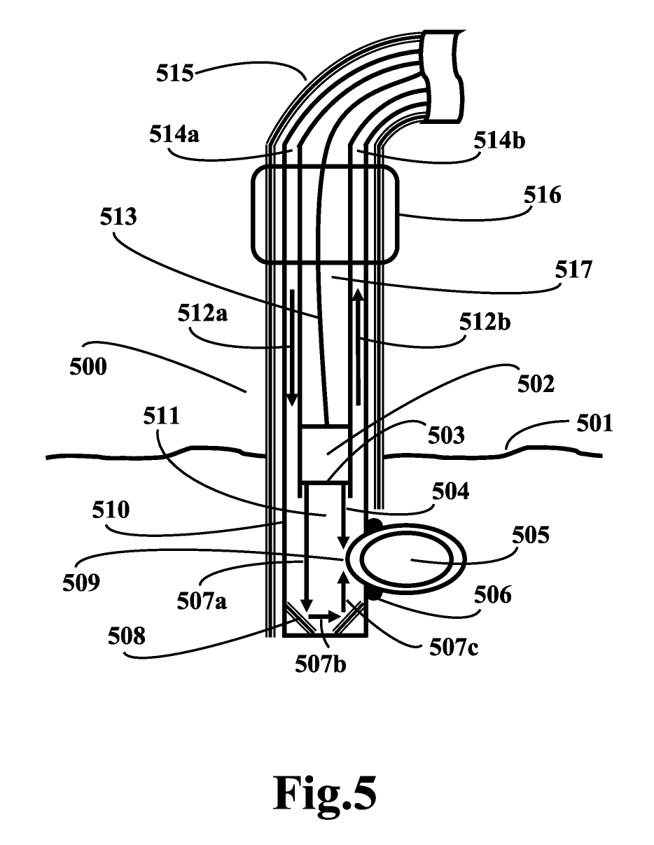

FIG. 5 disclose delivery device 500 used for minimal invasive procedures, for lateral removal of non-desired biological components from external surfaces of internally located tissues. The delivery system 500 is inserted via skin surface 501, using conventional guidance means such as catheter or for instance laparoscope-endoscope 515 having grasping handle 516. Housing 510 of device 500 include inflow tube 514a through which coupling and washing medium is inserted in the general direction of arrow 512a, and outflow tube 514b through which coupling and washing medium is removed in the general direction of arrow 512b. The outflow may be used also for the removal of biological components debris, removed from external surfaces of affected tissue. It shall be noted that coupling medium may be used also as washing medium. The housing 510 further contain dry zone 517, not exposed to ultrasound, as well as wet zone 511, in which ultrasound is emitted and ultrasound derived phenomena occur. Housing 510 is used to enable ultrasound derived phenomena at certain predetermined zones and angles. It therefore includes ultrasound emitting element 502 communicated via communication cable 513 and having emitting surface 503. The ultrasound emitting element 502 may also be located in upper zones, including closer to the control system so out of the body and distantly to skin 501, providing that acoustic wave-guide is provided enabling delivery of ultrasound of certain desired characteristics to wet zone 511. During procedure the inserted housing 510, that may be coupled with visualization means inserted via other channels of the laparoscope-endoscope 515, is placed in contact with external surfaces of desired internal tissue 505. Tissue 505 may for instance be tumor surface in solid place and then laparoscope-endoscope 515 is manipulated to reach target, however it may also be for instance portion of prostate removed during HOLEP procedure and floating in bladder, and then tissue may be manipulated toward laparoscope-endoscope 515. Manipulation of tissue may for instance be using suction, either per-se or via suctioning impact of outflow of 514b. Attachment to target is conducted via supporting element 506, being for instance rubber ring or ring with suction. For the actual removal of non-desired components, the ultrasound emitting element 502 is activated. Ultrasound waves propagate in general direction of arrows 504 and 507a, the latter being further reflected from ultrasound reflecting means 508, in general direction of arrows 507b and 507c. It is accordingly that ultrasound waves in directions of arrows 504 and 507c impinge the external surface of target 505 and affect for removal of biological component at area 509.

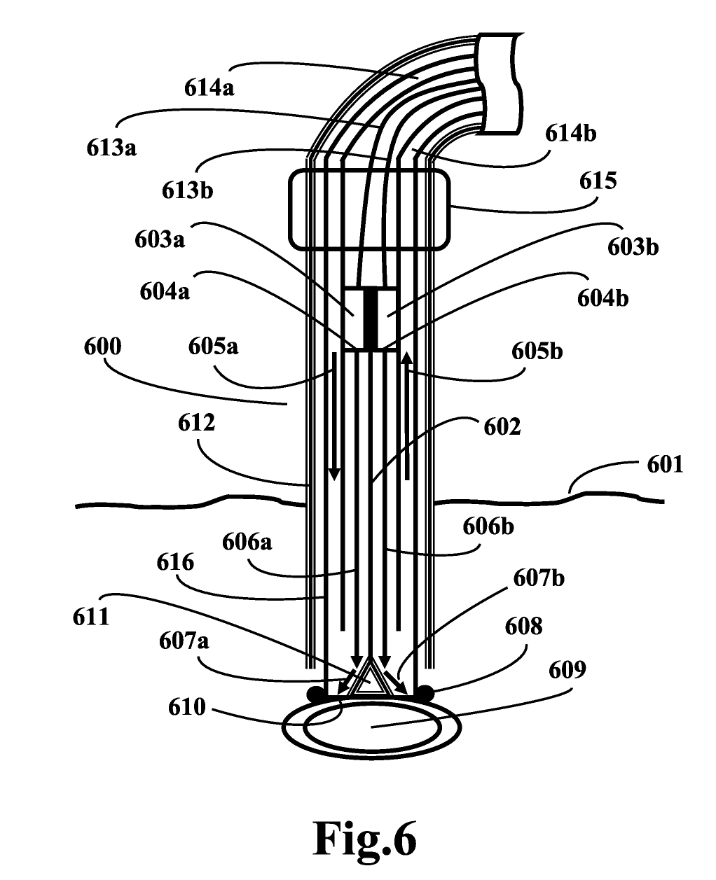

FIG. 6 disclose delivery device 600 used for minimal invasive procedures, for frontal removal of non-desired biological components from external surfaces of internally located tissues. The delivery system 600 is inserted via skin surface 601 using laparoscope-endoscope 612 having grasping handle 615. The system include ultrasound emitting elements 603a and 603b aligned at predetermined location and angle and being communicated to control unit via communication cables 613a and 613b, respectively. It shall be noted that ultrasound emitting elements may be located in the housing 616, or located closer to the control unit, providing ultrasonic guide with appropriate coupling means exist between emission source and target. The housing further incorporates inflow tube 614a and outflow tube 614b in which coupling medium is moved inwards and outwards in general direction of arrows 605a and 605b, respectively. The outward flow may be used to remove cellular debris detached from external surfaces of treated tissues. The housing 616 further include mechanical-supportive element 602, at end of which ultrasound reflecting element 611 having conical shape of predetermined reflecting angles is disposed. Procedure may be conducted while contacting target 609 via attachment means 608, rubber or suction elements. However if located in body cavity that includes coupling medium, for instance inside bladder having urine functioning as ultrasound contact medium, then there is no need for contact with surface of target 609 for the actual removal of biological components there from. During actuation, ultrasound waves propagate in the general direction of arrows 606a and 606b, impinge ultrasound reflecting element 611 and are being reflected and re-directed in general direction of arrows 607a and 607b to affect external surface 610 of target 609 for the removal of non-desired biological components therefrom.

While there have been shown preferred embodiments of ultrasonic methods and devices for the removal of non-desired biological components from external surfaces of tissues, it is to be understood that changes may be made therein without departing from the spirit of the invention. The invention embraces any and all changes, modifications, alternatives or rearrangements of the method and device.

1. A method for affecting for removal of biological components from external surfaces of tissues, using ultrasound derived mechanisms employed in coupling medium externally to the tissues, and while isolating the external tissues from the bulk of the affected ultrasound derived mechanisms.

2. A method according to claim 1, wherein the removal of biological components from external surfaces of tissues is carried out from the external surfaces inwards.

3. A method according to claim 2 wherein the biological components to be removed from external surfaces of tissues are integral components of the tissues.

4. A method according to claim 1, wherein ultrasound derived mechanisms employed are derived from ultrasound emitted at a certain predetermined angle.

5. A method according to claim 1, wherein coupling medium has been optimized to support related ultrasound derived mechanisms.

6. A device for affecting for removal of biological components from external surfaces of tissues, comprising:
At least one ultrasound emitting element adapted to emit ultrasound waves,
At least one supportive element adapted to enable ultrasound derived mechanisms demarcated in coupling medium,
Coupling medium adapted to support ultrasound derived mechanisms therein,
And while target-impinging ultrasound waves essentially do not create ultrasound derived mechanisms in target tissues.

7. A device according to claim 6, wherein the at least one ultrasound emitting element is adapted to create more than one ultrasound derived mechanisms.

8. A device according to claim 6, wherein the at least one ultrasound emitting element is adapted to create ultrasound derived mechanisms for affecting external surfaces of tissues using scanner.

9. A device according to claim 6, wherein supportive element enabling coupling medium demarcated ultrasound derived mechanisms is adapted to control angle of emitted ultrasound waves with respect to target.

10. A device according to claim 6, wherein supportive element enabling coupling medium demarcated ultrasound derived mechanisms is a ultrasound derived mechanism.

11. A device according to claim 6, and comprises also of filtration means adapted to remove from the coupling medium the biological components removed from the external surfaces.

12. A device according to claim 6, and comprises also of guiding means for minimal invasive procedures with the device of the invention.

13. A device according to claim 6, and comprises also of monitoring element

14. A device according to claim 13, wherein monitoring element used to feedback parameters of emitted ultrasound.

15. A device according to claim 6, wherein the coupling medium contain enrichment agents.
